SEQUENCE LISTING (35 sequences)

SEQ ID NO: 1 - protein, 7 aa, Artificial Sequence
  Kr5
PRKLYDY

SEQ ID NO: 2 - protein, 10 aa, Artificial Sequence
  Cilengitide (EMD 121974)
CRGDDFNMEV

SEQ ID NO: 3 - protein, 6 aa, Artificial Sequence
  Targeting RGD
CRGDHL

SEQ ID NO: 4 - protein, 9 aa, Artificial Sequence
  ATN-161
ACPHSCNNH

SEQ ID NO: 5 - protein, 25 aa, Artificial Sequence
  Tumstatin Peptide
TLPFAYCNIHQVCHYAQRNDRSYWL

SEQ ID NO: 6 - protein, 5 aa, Artificial Sequence
  Tumstatin fragment
YSNSG

SEQ ID NO: 7 - protein, 20 aa, Artificial Sequence
  Pentastatin-1
LRRFSTMPFMFCNINNVCNF

SEQ ID NO: 8 - protein, 27 aa, Artificial Sequence
  Endostatin peptide
HTHQDFQPVLHLVALNTPLSGGMRGIR

SEQ ID NO: 9 - protein, 50 aa, Artificial Sequence
  Endostatin fragment IV, IVox
CETWRTETTGATGQASSLLSGRLLEQKAASCHNSYIVLCIENSFMTSFSK

SEQ ID NO: 10 - protein, 20 aa, Artificial Sequence
  Endostatin peptide fragment I (180-199)
FLSSRLQDLYSIVRRADRAA

SEQ ID NO: 11 - protein, 12 aa, Artificial Sequence
  C16Y
DFKLFAVYIKYR

SEQ ID NO: 12 - protein, 12 aa, Artificial Sequence
  C16S
DFKLFAVTIKYR

SEQ ID NO: 13 - protein, 4 aa, Artificial Sequence
  VEGF derived peptide
DLPR

SEQ ID NO: 14 - protein, 20 aa, Artificial Sequence
  VEGF derived peptide 2
KSVRGKGKGQKRKRKKSRYK

SEQ ID NO: 15 - protein, 7 aa, Artificial Sequence
  FGF-derived peptide
ACARPCA

SEQ ID NO: 16 - protein, 14 aa, Artificial Sequence
  P144
TSLDASIIWAMMQN

SEQ ID NO: 17 - protein, 7 aa, Artificial Sequence
  Kr5
PRKLFDY

SEQ ID NO: 18 - protein, 8 aa, Artificial Sequence
  LAM
DFKLFAVY

SEQ ID NO: 19 - protein, 5 aa, Artificial Sequence
  HP
HHPHG

SEQ ID NO: 20 - protein, 7 aa, Artificial Sequence
  KLAK peptide
KLAKLAK

SEQ ID NO: 21 - protein, 8 aa, Artifical Sequence
  Modified KLAK
WKRAKLAK

SEQ ID NO: 22 - protein, 4 aa, Artificial Sequence
  FRAP-4
WEWT

SEQ ID NO: 23 - protein, 11 aa, Artificial Sequence
  DR-5 binding peptide
YCKVILTHRCY

SEQ ID NO: 24 - protein, 12 aa, Artificial Sequence
  TLS peptide
TLSGAFELSRDK

SEQ ID NO: 25 - protein, 16 aa, Artificial Sequence
  GO-203
RRRRRRRRRCQCRRKN

SEQ ID NO: 26 - protein, 24 aa, Artificial Sequence
  NOXA BH3
RRRRRRRRGECATQLRRFGDKLNF

SEQ ID NO: 27 - protein, 21 aa, Artificial Sequence
  NOXA mitochondrial targeting domain
RRRRRRRRGRQKLLNLISKLF

SEQ ID NO: 28 - protein, 20 aa, Artificial Sequence
  IP3R-derived peptide (IDP)
NVYTEIKCNSLLPLDDIVRV

SEQ ID NO: 29 - protein, 27 aa, Artificial Sequence
  LP-4 peptide
SWTWEKKLETAVNLAWTAGNSNKWTWK

SEQ ID NO: 30 - protein, 48 aa, Artificial Sequence
  TRAILmim/DR5
WDCLDNRIGRRQCVKLWDCLDNRIGKRQCVRLWDCLDNKIGRRQCVRL

SEQ ID NO: 31 - protein, 22 aa, Artificial Sequence
  p53-C terminal peptide
GSRAHSSHLKSKKGQSTSRHKK

SEQ ID NO: 32 - protein, 25 aa, Artificial Sequence
  CTMP4
LDPKLMKEEQMSQAQLFTRSFDDGL

SEQ ID NO: 33 - protein, 17 aa, Artificial Sequence
  Cationic lytic peptide
KLLLKLLKKLLKLLKKK

SEQ ID NO: 34 - protein, 16 aa, Artificial Sequence
  BH3 peptide
GQVGRQLAIIGDDINR

SEQ ID NO: 35 - protein, 9 aa, Artificial Sequence
  NuBCP-9 peptide
FSRSLHSLL

(19) United States
(12) Patent Application Publication
(10) Pub. No.: US 2019/0380957 A1
(43) Pub. Date: Dec. 19, 2019

(54) Protein Hydrogels For Treatment Of Neovascular Disease

(71) Applicant: New Jersey Institute of Technology, Newark, NJ (US)

(72) Inventors: Vivek A. Kumar, Newark, NJ (US); Peter Nguyen, New Rochelle, NY (US); Biplab Sarkar, Newark, NJ (US); Sruti Rachapudi, Houston, TX (US); Patricia Iglesias-Montoro, Haledon, NJ (US)

(73) Assignee: New Jersey Institute of Technology, Newark, NJ (US)

(21) Appl. No.: 16/442,044

(22) Filed: Jun. 14, 2019

Related U.S. Application Data
(60) Provisional application No. 62/685,468, filed on Jun. 15, 2018.

(51) Int. Cl. A61K 9/06; A61K 9/00; A61K 38/08; A61K 38/39; A61K 38/10; A61K 38/07; A61K 38/18; A61K 38/17

(52) U.S. Cl. CPC A61K 9/06; A61K 9/0048; A61K 9/0019; A61K 38/08; A61K 38/39; A61K 38/1758; A61K 38/07; A61K 38/1866; A61K 38/1761; A61K 38/1709; A61K 38/10

(57) ABSTRACT

A mimic of an anti-angiogenic peptide is combined with a self-assembling peptide hydrogel to provide improved treatment for pathological neovascularization management. Pathological neovascularization may cause or worsen intraocular posterior segment diseases, such as diabetic retinopathy (DR) and wet age-related macular degeneration (wet AMD). The attachment of a therapeutic anti-angiogenic motif to a fibrillizing peptide backbone that undergoes nanofibrous self-assembly into an injectable hydrogel was found beneficial for the treatment of aberrant neovascularization. The peptide persists for extended periods in a target site for prolonging the therapeutic timeframe. This injectable hydrogel therapy may unlock potential clinical routes for treating many neovascular diseases.

### CROSS-REFERENCE TO RELATED APPLICATIONS

The present application claims the benefit of the filing date of U.S. Provisional Patent Application No. 62/685,468 filed on Jun. 15, 2018 the disclosure of which is hereby incorporated herein by reference.

### FIELD OF USE

The present application discloses a functionalized peptide-based hydrogel that is an injectable material with anti-angiogenic capability and effect. More particularly, the present application relates to an anti-angiogenic sequence immobilized on a nanofibrous hydrogel to localize and prolong anti-angiogenic efficacy.

### BACKGROUND OF THE INVENTION

Angiogenesis is the formation of new blood vessels and is frequently associated with tumors or other pathological conditions. For example, pathological neovascularization of tissues can lead to a host of diseases like diabetic retinopathy (DR) and neovascular macular degeneration. Both of these conditions are intraocular posterior segment diseases that can be caused by formation of aberrant blood vessels in the retina and the choroid leading to progressive loss of visual acuity.

Approximately 10 percent of the total population in the United States has diabetes. Roughly one-quarter of those people will encounter some diabetic retinopathy according to the American Diabetes Association. Today, diabetic retinopathy is the most abundant form of eye disease in diabetic patients. DR is also the leading cause for blindness worldwide. It is estimated that blindness costs the U.S. Government approximately S13,607 annually per person in Social Security benefits, lost income tax revenue, and healthcare expenditures. If patients at risk for developing diabetic eye disease were regularly screened and then treated to preserve their sight, the net annual savings to the U.S. Government would be more than 100 million.

There is currently a lack of novel, minimally invasive, and effective therapy in the market for DR, since treatments often include ineffective injectables, laser procedures, or surgery. These treatments are insufficient in regards to both patient compliance and cost-effectiveness—for example, many patients report complications due to laser procedures. Diabetic retinopathy causes changes in the blood vessels of the retina. These blood vessels swell and leak. Additionally, numerous abnormal blood vessels grow on surface of retina obscuring vision and ultimately causing blindness. As a result of the severity of this eye disease and the magnitude of people affected, the necessity for treatment becomes quite clear.

Recent attempts to address this health need have been met with limited success. An anti-angiogenic peptide known as Kringle 5 (Kr5) has been shown to be a potent stimulator of endothelial cell (EC) apoptosis, but has poor bioavailability and non-specificity. Therapeutic anti-angiogenic agents are generally used in diffusible monomeric formulation (i.e., injection of anti-VEGF monoclonal antibodies into the vitreous humor). Anti-angiogenic peptides do provide an alternative to the standard-of-care antibody therapy. Several extracellular proteins have inherent anti-angiogenic activity. Short sequences in these proteins, such as Kringle (domain 5), Laminin-1, and Histidine-Proline-Rich-Glycoprotein, have been identified to be the active domains. However, these “mimic” sequences alone have limited in vivo efficacy as they diffuse away from the target site, limiting the window of efficacy and necessitating a large dosage.

Despite recent investigation of several promising therapeutic avenues, no therapies have been found for the safe and cost-effective treatment that leads to long-term attenuation of aberrant neovascularization to date.

Therefore, there still exists a critical need for a minimally invasive therapy to expunge such aberrant vascularization for a sustained period. There is also a need for a treatment that does not diffuse away from a target site and avoids limiting the window of efficacy and necessitating a large dosage.

### SUMMARY OF THE INVENTION

The present disclosure solves the problems of current state of the art and provides many more benefits. The composition and method of the present invention may be used in a variety of applications that involve over growth of vasculature, and treatment of neovasularization. The hydrogel specifically inhibits growth and proliferation of blood vessels. This composition has use in applications, such as tumor regression, wound healing modulation, and treatment of aberrant vascular growth on the retina—diabetic retinopathy, among other uses. It includes, but is not limited to, a functionalized peptide-based hydrogel that is injectable with anti-angiogenic capability.

In one embodiment, a new composition has an anti-angiogenic sequence PRKLYDY immobilized on a nanofibrous hydrogel to localize and prolong anti-angiogenic efficacy. The attachment of a therapeutic anti-angiogenic motif to a fibrillizing peptide backbone that undergoes nanofibrous self-assembly into an injectable hydrogel was found beneficial for the treatment of aberrant neovascularization. It was found that the peptide persists for extended periods in a target site for prolonging the therapeutic timeframe. The injectable hydrogel therapy is a potential clinical pathway for treating many neovascular diseases. Combining a mimic of this peptide with hydrogels that can be easily syringe aspirated, injected, and re-assemble in situ, provides a prolonged, sustained, and specific response for, among other things, DR management.

The ability for SLkr5 or K-(SL)₆-K-G-PRKLYDY or SL-Kr5, one new composition discussed herein, to be delivered topically, intravitreally, or locally to a treatment site is extremely beneficial. It further appeals to the market due to increased patient compliance when compared to invasive procedures, such as surgery. Therefore, one objective is to not only provide a novel treatment for DR, but to also prove an alternative cost effective and minimally invasive technique to treat DR and other pathological neovascularization.

Prevention of aberrant vascularization is thus an objective clinical target for the current disclosure. Since “mimic” sequences alone have limited in vivo efficacy as they can diffuse away from the target site, limiting the window of efficacy and necessitating a large dosage, this invention increases the efficacy period by attaching anti-angiogenic domains to self-assembled nanostructures, thus enabling high-density epitope domain presentation and preventing rapid dilution of the active species. Self-assembled peptide nanofibers with β-sheet fibrillizing domains have proven to be promising candidates for such delivery vehicles.

The fibrilizing domain consists of a peptide with polar or charged termini residues that flank an amphiphilic alternating hydrophilic/hydrophobic midblock. The composition of the fibrillizing domain may have a sequence of K-SLSLSLSLSLSL-K or K-(SL)₆-K.

As these peptides self-assemble into functionalized nanofibers and subsequently form hydrogels (at physiologic pH in aqueous buffer), the pendant domains are displayed at the fiber edges for receptor activation and related functionality. If these hydrogels are implanted in vivo, the cells in the surrounding fascia can attach to the hydrogel, infiltrate into the implant, and receive specifically designed cues from the functional domains. An injectable self-assembling peptide hydrogel (SAPH) platform may be used to immobilize a potent anti-angiogenic domain while retaining its efficacy, opening avenues for new effective anti-angiogenic therapeutics.

Another objective is to improve the current standard of care for DR by a minimally invasive topical delivery or intraocular injection of an anti-angiogenic drug that inhibits endothelial cell proliferation and migration rather than invasive techniques, such as laser surgeries. This objective is accomplished by using multi-domain peptides (MDPs), which are short amino acids sequences that self-assemble nanofibrous hydrogels. This affords thixotropic rheological properties—rapid shear thinning and shear recovery. Therefore, these hydrogels can be easily syringe aspirated, injected, and re-assemble in situ to provide a prolonged, sustained response.

Again, MDPs are short amino acids sequences with repeating hydrophobic and hydrophilic motifs that can be triggered to self-assemble in aqueous solution to form β-sheets and long-range nanofibers. Self-assembly is mediated by bonds that break and reassemble quickly: hydrogen bonding, Van der Waal's interactions, and ionic interactions. This affords thixotropic rheological properties—rapid shear thinning and shear recovery. Therefore, these hydrogels can be easily syringe aspirated, injected, and re-assemble in situ to provide a prolonged, sustained response, which has been evaluated for drug delivery. At the ultrastructural level, MDP self-assembles into large-scale extracellular matrix (ECM) mimetic nanofibers 2 nm thick, 6 nm wide, and several nm to 1 μm long. Injectable ECM mimetic scaffolds may rapidly infiltrate with cells that loaded drug can phenotypically modulate. Building upon in vivo drug release, a mimic of kringle5 (kr5) was engineered into the peptide sequence to allow for the development of hydrogels capable of endothelial cell apoptosis. These hydrogel moieties have shown reduction in vascular leakage in the retina of a diabetic mouse injected with streptozotocin (STZ). Current technologies for tissue engineering have attempted to capitalize on anti-angiogenic properties for reducing vasculature, and have limited success. However, the present invention has overcome these limitations.

A novel material is created to address neovascularization and disclosed herein. An 80 amino acid peptide known as kringle 5 (Kr5) has been shown to be a potent stimulator of EC apoptosis. A 7 amino acid mimic (PRKLYDY) of the active site of Kr5 has been shown to competitively bind to EC. Building on the synergies of the anti-angiogenic properties of the Kr5 mimic conjugated to injectable self-assembling peptides, a novel material is created that is now named (SLKr5). These peptides may be manufactured and assayed to significantly limit endothelial cell proliferation and migration.

In one embodiment, the attachment of an anti-angiogenic sequence (PRKLYDY) to the base peptide (K-SLSLSLSLSLSL-K) or K-(SL)₆-K is accomplished. The peptide mimic sequence attached to the base peptide derives from the extracellular plasminogen Kringle (domain 5). It has been previously shown that PRKLYDY can attach to endothelial cell surface protein GRP78 (glucose-regulated protein 78) and induce apoptosis of these endothelial cells. The small peptide (PRKLYDY) by itself would likely diffuse away quickly from the target site. A spacer such as G may be utilized in the attachement.

However, as discovered in the present invention, immobilization of the peptide on a nanofibrous hydrogel promotes anti-angiogenic efficacy of the sequence that allows the sequence to be localized and prolonged. Preliminary in situ response to MDP composition shows excellent optical transparency which supports a delivery of SL-Kr5 via topical eye drops or intraocular injection. MDPs moreover encourage two areas to be investigated: the ease with which they can be aspirated and the ability to re-assemble in situ to provide a prolonged, sustained response (such as material delivery).

Another advantage of the present invention is that the new composition MDP sequence contains three described domains: the termini (charged amino acid residues), the midblock (alternating hydrophobic and hydrophilic residues), and the signaling domain that may be altered. The invention may further utilize variations of the central self-assembling domain, terminal polar domains for self-assembly, related spacer, and changing the mimic in part or entirely to augment responses. Thus, numerous potential variations of the new composition are possible.

Still another objective is to study the effect that a mimic of the anti-angiogenic inhibitor Kr5 has on vascular endothelial growth factor (VEGF) expression to reduce vascular leakage. Together, the controlled addition of Kr5 to the self-assembling matrix offers a dynamic method for the first step of reducing vascular leakage to manage diabetic retinopathy (DR) and wet age-related macular degeneration (wet AMD).

The above objects and advantages are met by the present invention. In addition the above and yet other objects and advantages of the present invention will become apparent from the hereinafter-set forth Brief Description of the Drawings, Detailed Description of the Invention, and claims appended herewith. These features and other features are described and shown in the following drawings and detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

So that those having ordinary skill in the art will have a better understanding of how to make and use the disclosed composition and methods, reference is made to the accompanying figures wherein:

FIG. 1 is an HPLC trace of the novel peptide SL-Kr5 composition where purity of the peptide is >95%;

FIG. 2 is an electrospray ionization (ESI)-mass spectrum of the novel peptide SL-Kr5 tested in FIG. 1 having the expected average mass is 2509 Da; The [M+2H]²⁺ peak was observed at 1256.8 Da (expected at 1255.5 Da), the [M+3H]³⁺ peak was observed at 838.9 (expected at 837.3), and the [M+4H]⁴⁺ peak was observed at 628.3 Da (expected at 628.2);

FIGS. 3A-3C are illustrations showing self-assembly and secondary structure; FIG. 3A shows the various stages of self-assembly of the SL-Kr5 peptide and the formation of a β-sheet nanofiber to maximize canonical backbone hydrogen bonding to shield the hydrophobic amino acids in the core of the nanofiber; FIG. 3B shows a circular dichroism (CD) graph that indicates a β-sheet secondary structure of the nanofiber (characteristic minimum at 215 nm), and molar residual ellipticity was calculated as described in literature;

FIG. 3C shows a Fourier-transform infrared spectroscopy (FTIR) spectrum of the dried hydrogel that corroborates β-sheet conformation (amide I peak at 1624 cm⁻¹), and the 1673 cm⁻¹ peak has been attributed to lysine side-chains;

FIGS. 4A-4D illustrate biophysical characterization of the SAPH SL-Kr5, FIG. 4A shows a scanning electron microscopy (SEM) image of the critical-point dried hydrogel (inset: a photo of the hydrogel) that demonstrates the nanofibrous network underlying the hydrogel; FIG. 4B shows an atomic force microscopy (AFM) image of a diluted peptide solution (800 μM) that reveals individual constituent nanofibers (approximately 2 nm high); FIG. 4C shows a rheometric strain sweep of the hydrogel (1% to 100% strain, at a fixed oscillatory frequency 1 Hz) that shows the thixotropic (shear-thinning) property of the material; and FIG. 4D shows repeated strain cycles (1% strain and 100% strain) dynamically modulated the storage modulus of the nanofibrous hydrogel, even after repeated strain cycles, the resilient hydrogel can regain its viscoelastic properties within 10 seconds;

FIGS. 5A-5B shows a scanning electron microscopy image of hydrogels; FIG. 5A shows that critical point dried SL-Kr5 hydrogels display characteristic fibrous morphology associated with the self-assembling peptide hydrogel platform; FIG. 5B shows that uniformity in the nanofibrous structure provides confidence of a homogenous preparation at the surface and bulk;

FIG. 6 shows an additional atomic force microscopy image of the SL-Kr5 nanofibers;

FIGS. 7A-7B illustrates cytocompatibility of SL-Kr5; at relatively modest and high doses (8 μM (shown in FIG. 7A) and 80 μM) of SL-Kr5 in media, NIH 3T3 fibroblasts showed little to no difference in cytocompatibility compared to the media+sucrose control;

FIG. 7B illustrates at very high concentrations of 800 μM SL-Kr5 in media, a significant difference is seen in viability of cells from 85-90% to 55% (*p<0.05); this result supports our hypothesis that the peptide is specific for inhibition of endothelial cell proliferation and mitigates concern about possible off-site toxicity of the hydrogel;

FIGS. 8A-8F illustrate in vitro tube formation assay of human umbilical vein endothelial cells (HUVECs); FIG. 8A shows a negative control (sucrose in media) with EC tube formation; FIG. 8B shows tube formation is inhibited in the positive control (suramin in media);

FIGS. 8C-8E show that with higher concentrations of SL-Kr5 EC tube formation is inhibited; and FIG. 8F shows a quantitative comparison of the formulations tested for their inhibitory efficacy, assayed by the total length of EC tubes formed where different Greek letters denote statistically significant differences;

FIGS. 9A-9D illustrate results from a tube formation assay based quantitative evaluation of dose dependent SL-Kr5 inhibitory efficacy; the number of tube (FIG. 9A) branches, (FIG. 9B) junctions, (FIG. 9C) nodes, and (FIG. 9D) segments decreased with increasing concentrations of SL-Kr5; the highest concentration was comparable to the tested positive control (suramin), and an inhibitory efficacy increased with increasing peptide concentration;

FIGS. 10A-10D illustrate histology of SL-Kr5 implant; FIGS. 10A-10B show an ECM deposition around the implant on day 3 and day 7 (Masson's trichrome staining); and FIGS. 10C-10D magnified into the core of the hydrogel revealing minimal cellular infiltration with no obvious neovasculature or material degradation of the hydrogel (H&E staining);

FIGS. 11A-11B illustrate biocompatibility of scaffold implants; SL-Kr5 hydrogel was subcutaneously injected in the dorsal region of rats, and after 3 days the scaffolds were explanted and processed for histology; FIG. 11A shows H&E staining that reveals cells infiltrating the scaffold; and FIG. 11B magnifies the outer perimeter of the scaffold and illustrates the degradation of the hydrogel and new ECM deposition that occurred, which is in contrast to the lack of infiltration at the core of the implant;

FIGS. 12A-12C illustrate a comparison with similar self-assembled peptides (histology: H&E staining); FIG. 12A shows at 7 days that the core of the SL-Kr5 implant has much lower cellular infiltration compared to FIG. 12B which has K₂(SL)₆K₂; FIG. 12C is a SLanc hydrogel, which is a previously known composition by itself that has a VEGF-165 mimic appended, and it has significantly greater angiogenesis (arrows) compared to SL-Kr5, which has an anti-angiogenic mimic; large islands of under-graded hydrogels are easily observable in FIG. 12A compared to compositions in FIG. 12B and FIG. 12C, potentially due to muted canonical neovascularization in the periphery of biomaterial implants. Scale bar=100 μm; FIG. 12B and FIG. 12C both reproduced with permission from ACS and Elsevier, respectively.

### DETAILED DESCRIPTION

The present disclosure is directed to novel compositions and methods that overcome the drawbacks of current treatment methodologies for aberrant neovascularization. The current work described herein, among other things, how a functionalized peptide-based injectable biomaterial matrix operates as an effective implantable therapeutic. The development of a new anti-angiogenic self-assembling peptide, named SL-Kr5, allows for potential in vivo delivery and inhibition of aberrant neovascularization. The anti-angiogenic peptide hydrogel may be easily syringe aspirated, injected, and re-assembled in situ, which may provide prolonged, sustained, and tunable disease management. The current disclosure facilitates development and exploration of new therapeutic avenues to treat neovascular diseases, such as diabetic retinopathy, and potentially lead to site-directed therapeutics targeting tumor neovasculature.

It was found that the attachment of an anti-angiogenic sequence (PRKLYDY) to a fibrillizing domain (K-SLSLSLSLSLSL-K) by a glycine spacer or other such spacer may lead to a hybrid peptide that can self-assemble into a nanofibrous hydrogel and retain its anti-angiogenic functionality. When the spacer is a glycine spacer, it has 5 or less glycine spacers in the sequence. The peptide mimic sequence attached to the base peptide is derived from the extracellular plasminogen Kringle (domain 5). The sequence PRKLYDY can attach to endothelial cell surface protein GRP78 (glucose-regulated protein 78), inducing apoptosis of those cells. Again, the drawback is the small peptide (PRKLYDY), by itself, may diffuse away quickly from the target site. One solution to this diffusion issue, proposed by this present disclosure, is that the anti-angiogenic efficacy of the domain can be localized and prolonged when it is immobilized in a nanofibrous hydrogel.

The following example(s) illustrate the features of the invention. In no way is the following example meant to limit the scope of the invention to a particular embodiment. The example is merely given as one aspect of the invention and to illustrate its desired properties.

### Example 1

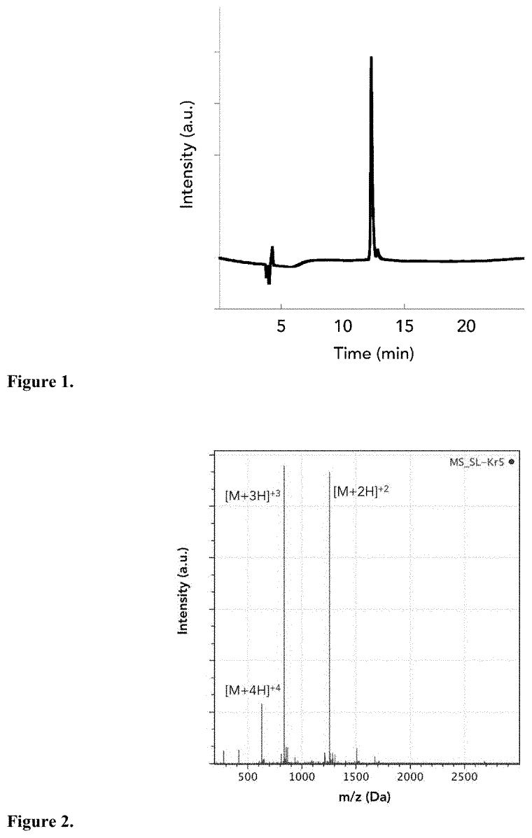

Synthesized, among other things, was the novel target peptide, SL-Kr5 (K-(SL)₆-K-G-PRKLYDY), through Fmoc solid-phase peptide chemistry, purified by HPLC (FIG. 1) and dialysis, and identified it by ESI mass spectrometry as shown in FIG. 2. The aqueous peptide solution was then lyophilized into dried powder form. The peptide forms a viscoelastic hydrogel when the dried peptide is dissolved (8 mM peptide in 298 mM sucrose solution at pH 7).

It was hypothesized that the hybrid peptide would undergo nanofibrous self-assembly, facilitated by its central fibrillizing domain, [(SL)₆], with alternating hydrophilic and hydrophobic residues as shown in FIG. 3A. The high propensity of the central domain to form β-sheet nanofibers has been attributed to hydrophobic interactions among the leucine residues as well as the canonical β-sheet backbone hydrogen bonding also shown in FIG. 3A. The predicted secondary structure of the nanofibers is confirmed in solution phase (8 μM solution) by CD spectroscopy and in dried hydrogel form by FTIR spectroscopy. The minimum at 215 nm in CD shown in FIG. 3B and the peak at 1624 cm⁻¹ in FTIR shown in FIG. 3C are signatures of a β-sheet secondary conformation.

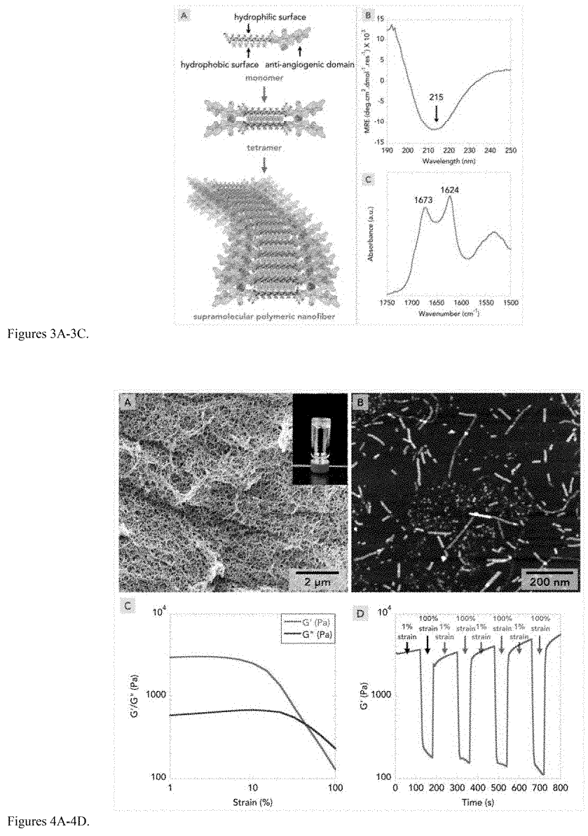

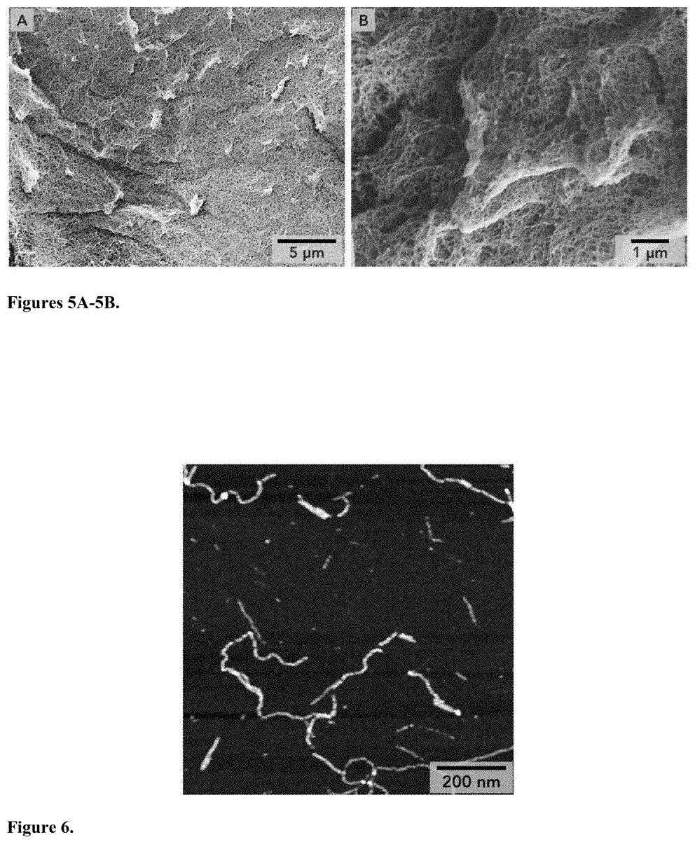

Critical-point dried samples of the hydrogel reveal a mesh-like nanofibrous architecture in SEM (FIGS. 4A, 5A-5B). The individual peptide strands can be identified in AFM (FIG. 4B, FIG. 6). The nanofibers were ˜2 nm high and ˜13 nm wide. The nanofibrous hydrogel demonstrated shear-thinning, i.e., it liquefied at high shear strain (FIG. 4C). The hydrogel was also able to recover its storage modulus within 10 s of strain removal (FIG. 4D).

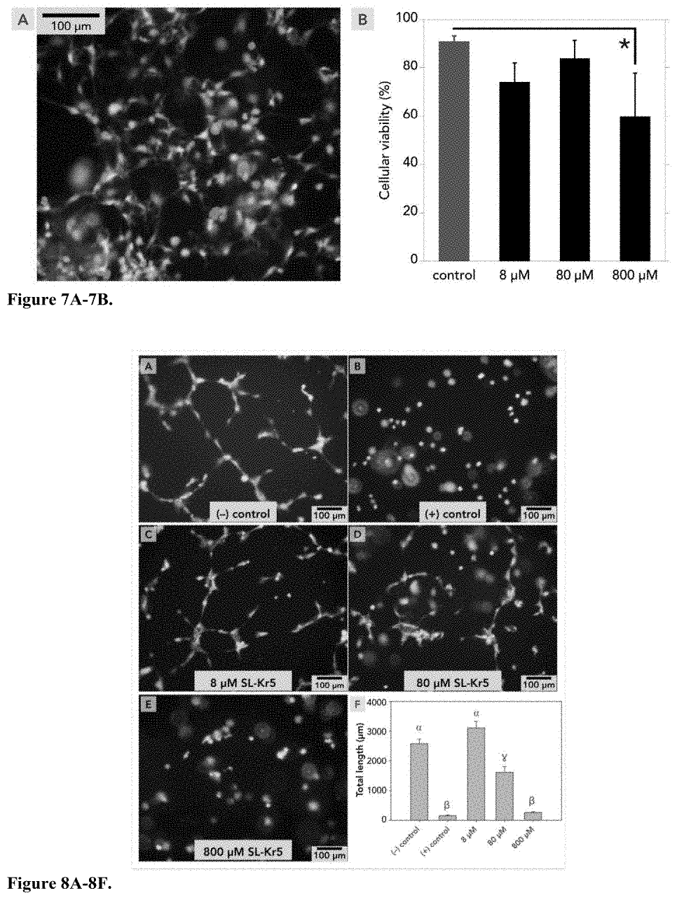

Cytocompatibility was determined by fibroblast cultures treated with increasing concentrations of the peptide in culture media. SL-Kr5 showed comparable cellular proliferation to controls over a range of concentrations (FIGS. 7A-7B). Thus, the peptide did not induce non-specific toxicity in stromal cells, which is in contrast to the expected inhibitory effect of the peptide on endothelial cells.

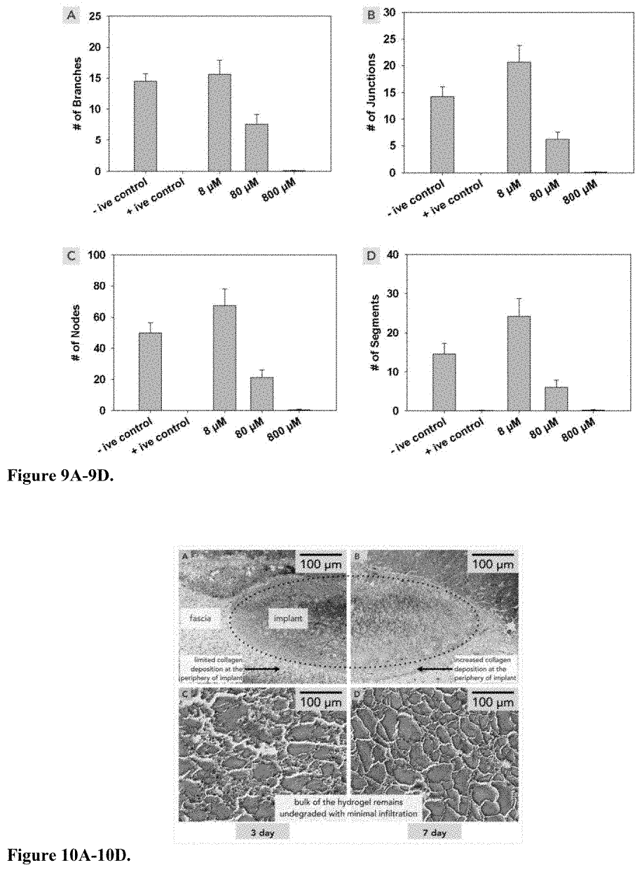

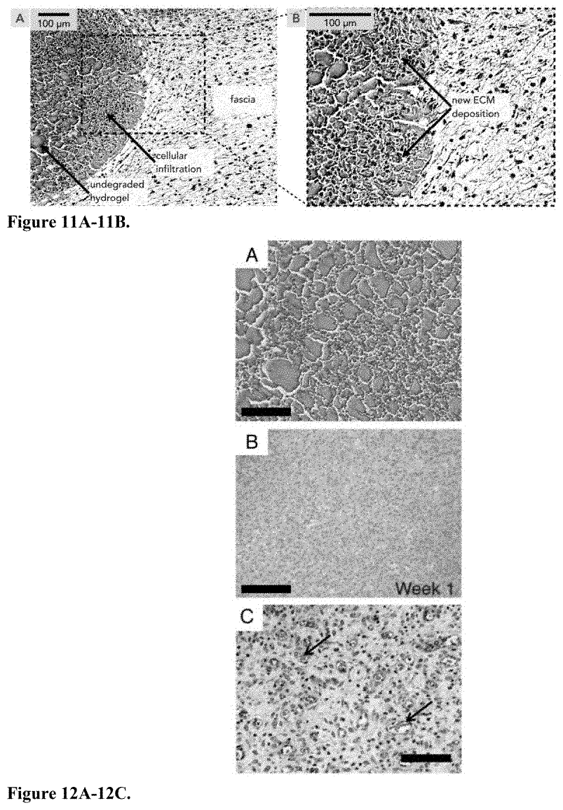

To assay the anti-angiogenic effect of the self-assembled peptide on endothelial cells, tube formation assays were performed for a range of peptide concentrations. This assay captures the propensity of human ECs or HUVECs to form capillary-like tubules. The process is sensitive to the presence of anti-angiogenic compounds. HUVECs were seeded along with varying concentrations of SL-Kr5 solution on a basement membrane extract matrix to stimulate the formation of capillary tubules. The effects of the peptide on the HUVECs were characterized by measuring the extent of tube formation in terms of total length of tubules formed, number of segments, number of branches, number of junctions, number of nodes, and number of segments (FIGS. 8A-8F, FIGS. 9A-9D). Media was used supplemented with sucrose as the negative control and another supplemented with suramin (a known anti-angiogenic compound) as the positive control for the inhibition of tube formation by different concentrations (8 μM, 80 μM, 800 μM) of SL-Kr5. Detection methods were able to detect a dose-dependent inhibitory effect of SL-Kr5 on tube formation and angiogenesis (FIGS. 8A-8F, FIGS. 9A-9D) where a higher dosage of SL-Kr5 progressively inhibited EC tube formation. Combined with the fibroblast cytocompatibility data, the tube formation assay conclusively demonstrated the anti-angiogenic efficacy and specificity of SL-Kr5. Rat dorsal subcutaneous implantation was used to study the in vivo physiological response towards SL-Kr5. 200 μL SC implants were injected and retrieved at prescribed time points (3 days and 7 days) for histological assessment. The implants demonstrated that the SAPH formulation was easily injectable with a 30 gauge syringe needle, offering no more appreciable resistance than saline. At 3 days, there was very limited ECM deposition surrounding the implant whereas at 7 days substantial deposition of collagen was seen on the periphery of the hydrogel implant (FIGS. 10A-10B). Similar to other SAPHs, there was no fibrous capsule apparent as evidenced by the absence of microvessels and high cell density around the perimeter of the implant. The implants allowed limited infiltration of host cells from the surrounding fascia into the periphery of the scaffold (FIGS. 11A-11B). The cellular infiltration was significantly muted when compared to similar SAPHs, and assessment of the core of the hydrogel showed especially limited infiltration and material degradation (FIGS. 10C-10D, FIGS. 12A-12C). Although 200 μL was utilized in this example, it is contemplated that an administered dosage of SL-Kr5 to patients in an amount of about 5 μL-1,000 μL may be used and preferably about 5 μL-200 μL. It is further contemplated that an administered concentration of SL-Kr5 about 0.2 μM-20,000 μM may be used and preferably about 8 μM-800 μM.

Compared to other SAPHs with or without attached angiogenic moiety, SL-Kr5 resulted in significantly slower scaffold degradation, less cellular infiltration, and no angiogenesis or neurogenesis (FIGS. 12A-12C). This result was attributed to the recapitulation of scaffold-based signaling activity of the mimic, since it prevents formation of neovasculature in the implant or immediate vicinity, limiting immune cell infiltration.

It is instructive to compare SL-Kr5 to the previously reported self-assembling peptide SLanc (FIG. 12A-12C). SLanc also contains a β-sheet fibrillizing domain similar to SL-Kr5. It contains an additional MMP2 cleavable site inside the fibrillizing domain. It also contains an angiogenic domain that mimics VEGF-165 and has been shown in subcutaneous implantation studies to not only rapidly recruit immune cells from the surrounding fascia, but also to become highly neovascularized on day 7 (FIGS. 12A-12C), which is in stark contrast to SL-Kr5 (FIG. 10D). The combination of SLanc and SL-Kr5 provides us a pair of “on/off” signals for angiogenesis. Such promoter/inhibitor pairs may become increasingly crucial for constructing next-generation multicomponent tissue-engineered scaffolds with spatiotemporally patterned cellular niches.

The slower degradation of SL-Kr5 in comparison to SLanc may be attributed to the absence of any enzymatic cleavage domain in its sequence. A slow degradation profile is desirable for in vivo applications, such as intraocular implantation for managing diabetic retinopathy. In addition, the present disclosure's novel slower degradation profile is desirable for in vivo applications, not only for intraocular implantation for managing diabetic retinopathy, but also useful to treat macular edema, age-related macular degeneration, proliferative eye disease, proliferate neovascular disease, and prevention of neovasculature formation in an implant or immediate vicinity to limit immune cell infiltration.

In fact, the self-assembling peptide K₂(SL)₆K₂, which contains the same fibrillizing domain as SL-Kr5 and has no MMP-cleavable sites, can undergo slow in vivo biodegradation over 6 weeks. It was noted in this Example testing that the lack of neovasculature in SL-Kr5 implants cannot simply be attributed to the lack of a VEGF-mimic sequence present in SLanc because K₂(SL)₆K₂, which does not contain any such mimic, has demonstrated a strong basal level of angiogenesis. Thus, it was ascribed that the lack of angiogenesis in the SL-Kr5 implants are to be potentiated by the Kringle (domain 5) mimic, which is consistent with our in vitro results (FIG. 8A-8F).

In addition to the covalently attached anti-angiogenic functionality, it may be possible to exploit the synergy of non-covalent storage of steroids, such as dexamethasone and triamcinolone acetonide, or anti-VEGF drugs within the hydrogel. In this scenario, after the initial release of the sequestered drug, the anti-angiogenic peptide itself may slowly dissociate from the nanofibrous scaffold and may induce apoptosis of the endothelial cells in the targeted tissue niche. Drug delivery potential of the SAPH platform has been demonstrated previously for the release of both small molecules (i.e., suramin) as well as large biomolecules (i.e., IL-4). Long term delivery of drugs in vivo is limited by scaffold degradation. Notwithstanding the combinatorial strategies available, a scaffold that is intrinsically anti-angiogenic with a slow rate of degradation may be useful for the management of neovascular pathologies.

### Example 1 Experimental Methods

Peptide Synthesis and Purification: SL-Kr5 was synthesized by solid-phase peptide synthesis with acetyl N-terminal and amide C-terminal protective groups, using previously known methods. The peptide was dissolved in DI water and purified by HPLC (FIG. 1). Next, the peptide solution was dialyzed against DI water in 2 kDa molecular weight cut-off dialysis tubing. The peptide was then identified by ESI mass spectrometry (FIG. 2). The peptide was stored in a lyophilized form.

Peptide Characterization: CD, SEM, AFM, FTIR, and rheology methods were performed under standard methodology. Circular dichroism was performed using an Olis Rapid Scanning Monochromator to measure the ellipticity of a 0.002% (w/v) peptide solution from 190 nm to 240 nm in a 1 cm cuvette. The ellipticity was then converted to molar residual ellipticity, [θ], according to the following known formula:

$$[ θ ] = \frac{θ \times m}{10 \times c \times l \times n}$$

where θ is ellipticity, m is the molecular weight of the peptide, c is the concentration of the peptide solution in mg/mL, 1 is the path length of the cuvette in cm, and n is the number of residues in the peptide sequence.

To perform SEM, hydrogel samples of SL-Kr5 were fixed in 2% glutaraldehyde, ethanol dehydrated, and critical-point dried. Samples were then sputter-coated with 8 nm gold/palladium and imaged using a LEO 1530VP Field Emission SEM at a working distance of ˜10 mm.

AFM was performed on diluted (0.2% w/v) peptide hydrogels deposited, spin-coated, and air dried on a freshly cleaved mica disc. PeakForce Tapping (ScanAsyst) mode was used on a Bruker Dimension Icon AFM.

For rheology, a 2% (w/v) peptide hydrogel was transferred between a 4 mm parallel plate geometry and a plate. The gap was set to 250 μm. Strain sweep (1-100% strain at 1 rad/s) and repeated shear recovery (1% strain at 1 Hz and 100% strain at 1 Hz, 4 cycles) were performed using a Malvern Kinexus Ultra+ rheometer.

For FTIR, 2% (w/v) peptide hydrogels were transferred onto an attenuated total reflectance accessory and air-dried into a thin film. Infrared spectra between 400 cm⁻¹ and 4000 cm⁻¹ were collected using a PerkinElmer Spectrum 100 FTIR spectrometer.

In Vitro Cytocompatibility: NIH 3T3 fibroblasts were cultured in media (Dulbecco's Modified Eagle Medium supplemented with 10% fetal bovine serum and 1% 100× penicillin streptomycin) in T75 flasks. After the cells reached confluency, they were seeded in a 96-well plate at 2,500 cells/well. Three target conditions (800 μM, 80 μM, and 8 SL-Kr5; n=6) and a control (media with sucrose; n=6) were tested. For target conditions, the peptide was supplemented in the media, and for the media control, 298 mM sucrose (formulation carrier) was added. Media was changed daily and cytocompatibility was assessed on day 3 using a LIVE/DEAD® viability/cytotoxicity kit. Images were taken on a Nikon Instruments Eclipse Ti-S inverted fluorescence microscope, and cell viability was quantified using NIH ImageJ, a known image analysis and processing software program.

Tube Formation Assay: HUVECs (Lonza) were cultured in T75 flasks using an Endothelial Cell Growth Medium BulletKit (Lonza). A Cultrex In Vitro Angiogenesis Assay Kit was used to measure tube formation. All steps were performed according to the manufacturer's instructions, which are briefly described as follows. 50 μL of basement membrane extract was added to each well of a 96-well plate and incubated at 37° C. for 30 to 60 min. Confluent HUVECs were stained with 2 μM calcein AM for 30 min at 37° C. and seeded on the basement membrane extract in the 96-well plate at 3,000 cells/well. Three target conditions (800 μM SL-Kr5, 80 μM SL-Kr5, and 8 μM SL-Kr5; n=6) and two controls (media with sucrose and media with suramin; n=6) were tested. The negative control was supplemented with 298 mM sucrose to match the corresponding highest quantity added to the test conditions, and the positive control contained 1.8 mM suramin. The 96-well plate was then incubated at 37° C. for 4 to 6 hours before imaging on a Nikon Instruments Eclipse Ti-S inverted fluorescence microscope. Tube length was quantified using NIH ImageJ software.

In Vivo Subcutaneous Implantation: All animal studies were approved by the NJIT-Rutgers institutional animal care and use committee. Female Wistar rats (225-250 g, Charles River Labs) were prepped and injected subcutaneously in the dorsal region with 200 μL of SL-Kr5 hydrogel (n=4) and two time points (3 day and 7 day). At the specified time points, the rats were sacrificed and regions around the implant were excised, fixed, and processed to 8 μm paraffin sections for routine histology staining and analysis (H&E and Masson's trichrome).

Statistical Analysis: For multiple comparisons, ANOVA was used with Tukey post hoc analysis for parametric data. Any nonparametric data was evaluated using the Kruskal-Wallis ANOVA with Dunn's post hoc analysis. Statistical significance was accepted for p<0.05.

As a result of the above experimentation, it was found that the attachment of a therapeutic anti-angiogenic motif to a fibrillizing peptide backbone that undergoes nanofibrous self-assembly into an injectable hydrogel was beneficial for the treatment of aberrant neovascularization.

The peptide persists for extended periods in a target site for prolonging the therapeutic timeframe. This injectable hydrogel therapy may unlock potential clinical routes for treating many neovascular diseases.

In summary, the attachment of an anti-angiogenic sequence, such as but not limited to the illustrated PRKLYDY polypeptide to a fibrillizing domain, including but not limited to K-SLSLSLSLSLSL-K by a spacer like glycine leads to a hybrid peptide. This hybrid peptide can self-assemble into a nanofibrous hydrogel and retain anti-angiogenic functionality unlike other peptides that are not in a hydrogel. And unlike a small peptide alone, namely PRKLYDY, by itself that diffuses away quickly from a target site. The antiangiogenic efficacy of the domain peptide is localized and prolonged when it is immobilized in the hydrogel, namely the nanofibrous hydrogel.

Other antiangiogenic and proapoptotic sequences may be utilized instead of PRKLYDY. The anti-angiogenic peptide domain sequence is a short mimic epitope of a larger protein growth factor, cytokine, chemokine, signaling molecule that promotes the disruption in signaling, network formation or the apoptosis of endothelial cells.

Below in Table 1 are the proposed mimics or sequences and a sampling of other antiangiogenic and proapoptotic sequences that may be utilized with the proposed sequence K-(SL)₆-K-G- instead of PRKLYDY. Sequences [1-19] contain antiangiogenic sequences. Sequences [20-35] contain proapoptotic sequences. Again any of these sequences in Table 1 may be attached to K-(SL)₆-K-G- or K-SLSLSLSLSLSL-K-G- instead of the PRKLYDY protein that forms K-(SL)₆-K-G-PRKLYDY or new hybrid protein SL-Kr5.

TABLE 1
Listing of Sequences that Attach to K-(SL)₆-K-G-

| | | | Origin |
|---|---|---|---|
| SEQ. NO | Name | Sequence | Protein |
| SEQ. NO 1 | unknown | PRKLYDY | unknown |
| SEQ. NO 2 | Cilengitide | c-[RGD-DF-NMEV] | RGD, |
| | (EMD 121974) | | Fibronectin |
| SEQ. NO 3 | Targeting RGD | cRGD-HL | Collagen IV |
| SEQ. NO 4 | ATN-161 | Ac-PHSCN-NH2 | Fibronectin |
| SEQ. NO 5 | Tumstatin | TLPFAYCNIHQV | Collagen IV |
| | Peptide | CHYAQRNDRSY WL | |
| SEQ. NO 6 | Tumstatin | YSNSG | Collagen IV |
| | fragment | | |
| SEQ. NO 7 | Pentastatin-1 | LRRFSTMPFMFCNIN | Collagen IV |
| | | NVCNF | |
| SEQ. NO 8 | Endostatin | HTHQDFQPVLHLVA | Collagen |
| | peptide | LNTPLSGGMR GIR | XVIII |
| SEQ. NO 9 | Endostatin | CETWRTETTGATGQ | Collagen |
| | fragment IV, | ASSLLSGRLLEQKA | XVIII |
| | IVox | ASCHNSYIVLCIENS | |
| | | FMTSFSK | |
| SEQ. NO 10 | Endostatin | FLSSRLQDLYSIVRR | Collagen |
| | peptide | ADRAA (20) | XVIII |
| | fragment I (180- | | |
| | 199) | | |
| SEQ. NO 11 | C16Y | DFKLFAVYIKYR | Laminin |
| SEQ. NO 12 | C16S | DFKLFAVTIKYR | Laminin |
| SEQ. NO 13 | VEGF derived | D^{(LPR)} | VEGF-B |
| | peptide | | |
| SEQ. NO 14 | VEGF derived | KSVRGKGKGQKRK | Exon 6a of |
| | peptide 2 | RKKSRYK | VEGF gene |
| SEQ. NO 15 | FGF-derived | Ac-ARPCA | PTX3 |
| | peptide | | |
| SEQ. NO 16 | P144 | TSLDASIIWAMMQN | TGFβ |
| | | | Receptor |
| SEQ. NO 17 | Kr5 | PRKLFDY | Kringle 5 |
| SEQ. NO 18 | LAM | DFKLFAVY | Laminin-1 |
| SEQ. NO 19 | HP | (HHPHG)4 | Histidine- |
| | | | proline-rich |
| | | | glycoprotein |
| SEQ. NO 20 | KLAK peptide | (KLAKLAK)2 | De novo |
| SEQ. NO 21 | Modified KLAK | WKRAKLAK | Modified |
| | | | KLAK |
| | | | peptide |
| SEQ. NO 22 | FRAP-4 | WEWT | FasL |
| SEQ. NO 23 | DR-5 binding | YCKVILTHRCY | DR5 |
| | peptide | | |
| SEQ. NO 24 | TLS peptide | TLSGAFELSRDK | Bcl-2 |
| SEQ. NO 25 | GO-203 | RRRRRRRRRCQCRR | MUC1-C |
| | | KN | |
| SEQ. NO 26 | NOXA BH3 | RRRRRRRRGECATQ | NOXA |
| | | LRRFGDKLNF | |
| SEQ. NO 27 | NOXA | RRRRRRRRGRQKLL | NOXA |
| | mitochondrial | NLISKLF | |
| | targeting | | |
| | domain | | |
| SEQ. NO 28 | IP3R-derived | NVYTEIKCNSLLPLD | IP3R |
| | peptide (IDP) | DIVRV | |
| SEQ. NO 29 | LP-4 peptide | SWTWEKKLETAVN | VDAC1 |
| | | LAWTAGNSNKWTW | |
| | | K | |
| SEQ. NO 30 | TRAIL^{mim/DR5} | WDCLDNRIGRRQCV | TRAIL |
| | | KL | |
| | | WDCLDNRIGKRQCV | |
| | | RL | |
| | | WDCLDNKIGRRQCV | |
| | | RL | |
| SEQ. NO 31 | p53-C terminal | GSRAHSSHLKSKKG | TP53 |
| | peptide | QSTSRHKK | |
| SEQ. NO 32 | CTMP4 | LDPKLMKEEQMSQ | CTMP |
| | | AQLFTRSFDDGL | |
| SEQ. NO 33 | Cationic lytic | KLLLKLLKKLLKLL | EGFR |
| | peptide | KKK | |
| SEQ. NO 34 | BH3 peptide | GQVGRQLAIIGDDIN | LHRH/(BH3 |
| | | R | |
| SEQ. NO 35 | NuBCP-9 | FSRSLHSLL | Nur77 |
| | peptide | | |

The protein is relatively short where the hybrid peptide contains between about 5-50 amino acids. This short sequence contains active domains. In the past, these type of “mimic” sequences alone have had limited in vivo efficacy since they readily diffuse away from a targeted site, thus limiting the efficacy time and requiring much larger dosages. The use of the self assembling hydrogel avoids these drawbacks and requires less of a dosage than current methodologies.

The hybrid peptide has a much slower degradation than mother self assembling peptides that form hydrogels. For example the slower degradation of SL-Kr5 in comparison to SLanc is attributed to the absence of an enzymatic cleavage domain in the SL-Kr5 sequence. Again, a slow degradation profile is desirable for in vivo applications, such as, but not limited to, intraocular implantation for managing diabetic retinopathy.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

### ABBREVIATIONS

CD, circular dichroism;
SEM, scanning electron microscopy;
AFM, atomic force microscopy;
FTIR, Fourier-transform infrared spectroscopy;
SAPH, self-assembling peptide hydrogel;
EC, endothelial cell;
HUVEC, human umbilical vein endothelial cell;
ECM, extracellular matrix;
VEGF, vascular endothelial growth factor;
MMP, matrix metalloprotease;
HPLC, high-performance liquid chromatography;
ESI, electrospray ionization;
DI, deionized;
H&E, hematoxylin and eosin.

What is claimed is:

1. A composition for management of a neovascular pathology, comprising
an anti-angiogenic or proapoptotic peptide domain sequence attached to a fibrillizing domain by a spacer to form a hybrid peptide;
a hydrogel formed by the hybrid peptide that is self-assembling; and
wherein the peptide domain sequence is immobilized in the hydrogel to localize and prolong anti-angiogenic or proapoptotic efficacy for management of a neovascular pathology and prevention of neovasculature formation.

2. The composition of claim 1, wherein the hybrid peptide contains between about 5 to 50 amino acids.

3. The composition of claim 1, wherein the peptide domain is a short mimic epitope of a larger protein growth factor, cytokine, chemokine, signaling molecule that promotes disruption in signaling, network formation, or apoptosis of endothelial cells.

4. The composition of claim 3, wherein the mimic includes PRKLYDY, or a peptide from [SEQ. 2]-[SEQ. 35].

5. The composition in claim 1, wherein the fibrilizing domain consists of a peptide with polar or charged termini residues that flank an amphiphilic alternating hydrophilic and/or hydrophobic midblock.

6. The composition in claim 1, wherein the fibrilizing domain is K-SLSLSLSLSLSL-K.

7. The composition in claim 1, wherein the hybrid peptide is K-(SL)₆-K-G-PRKLYDY or SL-Kr5.

8. The composition in claim 1, wherein the spacer is a glycine spacer in an amount of six or less of the glycine spacer.

9. The composition in claim 1, wherein the fibrillizing domain is K-SLSLSLSLSLSL-K connected to the anti-angiogenic peptide domain sequence that is PRKLYDY by the spacer that is a glycine spacer to form the hybrid peptide that is K-(SL)₆-K-G-PRKLYDY or SL-Kr5.

10. The composition of claim 1, wherein the hydrogel is a nanofibrous hydrogel, and the nanofibrous hydrogel prolongs antiangiogenic efficacy of the domain sequence.

11. The composition of claim 1, wherein the hydrogel is a biodegradable and an injectable hydrogel.

12. The composition of claim 1, wherein the hybrid peptide is K-(SL)₆-K-G-PRKLYDY or SL-Kr5; and the hybrid peptide has a slower degradation profile than at least one other self-assembling peptide attributed to absence of an enzymatic cleavage domain in the hybrid peptide.

13. The composition of claim 12, wherein, the slower degradation profile is desirable for in vivo applications, such as intraocular implantation for managing diabetic retinopathy, macular edema, age-related macular degeneration, proliferative eye disease, proliferate neovascular disease, and prevention of neovasculature formation in an implant or immediate vicinity to limit immune cell infiltration.

14. A method of administering a composition for management of a neovascular pathology, comprising
administering a composition having a multidomain peptide (MDP) composition of SL-Kr5 or (K-(SL)₆-K-G-PRKLYDY) for management of a neovascular pathology.

15. The method of claim 14, wherein the neovascular pathology includes an attenuation of aberrant neovascularization found in diabetic retinopathy (DR).

16. The method of claim 14, wherein the composition administration is done topically, intravitreally, or locally to a treatment site.

17. The method of claim 14, wherein an administered dosage of SL-Kr5 is in an amount of about 5 μL-1000 μL.

18. The method of claim 14, wherein an administered concentration of SL-Kr5 is about 0.2 μM-20,000 μM.

19. A method of synthesizing a composition for management of neovascular pathology, comprising,
synthesizing a hybrid peptide, SL-Kr5 or (K-(SL)₆-K-G-PRKLYDY) through Fmoc solid phase peptide synthesis;
purifying the hybrid peptide by HPLC and dialysis to form a liquid aqueous peptide solution;
lyophilizing the liquid aqueous peptide solution to a dried peptide powder;
dissolving the dried peptide powder in a sucrose solution to undergo a nanofibrous self-assembly facilitated by a central fibrillizing domain (SL)₆ having alternating hydrophilic and hydrophobic residues; and
forming a viscoelastic hydrogel or a self-assembled hydrogel that retains anti-angiogenic functionality for management of a neovascular pathology.

20. The method of claim 19, wherein the synthesizing further includes attaching an anti-angiogenic sequence PRKLYDY to a fibrillizing domain K-(SL)₆-K by a glycine spacer to form the self-assembled hydrogel.

21. The method of claim 19, wherein 8 mM of the dried peptide powder is dissolved in 298 mM the sucrose solution at a pH 7.

22. The method of claim 19, wherein the neovascular pathology is selected from a group consisting of diabetic retinopathy (DR), intraocular posterior segment diseases, cancerous tumor growth, age-related macular degeneration (AMD), and any combination thereof.
